(19) United States
(12) Patent Application Publication
(10) Pub. No.: US 2004/0181267 A1
(43) Pub. Date: Sep. 16, 2004

(54) Laser therapy device for the treatment of skin diseases

(76) Inventor: Hans Michael Ockenfels, Alzenau (DE)

Correspondence Address: ALTERA LAW GROUP, LLC, 6500 CITY WEST PARKWAY, SUITE 100, MINNEAPOLIS, MN, 55344-7704, US

(21) Appl. No.: 10/689,463

(22) Filed: Oct. 20, 2003

(30) Foreign Application Priority Data
Mar. 13, 2003 (DE) 103 10 913.7

(51) Int. Cl. A61N 5/06

(52) U.S. Cl. 607/89; 607/94

(57) ABSTRACT

The invention relates to a method for the treatment of skin diseases, in particular psoriasis, by means of UV radiation generated by a laser and directed towards the skin areas affected by the skin disease. Such method provides that the thickness of the epidermis in the skin areas is determined and the laser radiation dose regulated depending on the epidermis thickness so detected. The invention is based on the knowledge that the effect of UV radiation on the affected skin areas where psoriasis has developed into so-called plaques is primarily governed by the thickness of the epidermis within such plaques and not by the skin type or the MED (minimal erythema dose) established for healthy skin regions. For the determination of the thickness of the epidermis an ultrasonic device is preferably employed. As a result of the laser radiation dose being adapted to the individual patient and individual plaque the treatment objective is achieved earlier with the total radiation dose being reduced and less side effects occurring than experienced with comparable treatment methods known from the state of the art. Furthermore, the invention relates to a laser therapy device for the implementation of the method proposed according to the invention.

The invention relates to a method and a laser therapy device for the treatment of skin diseases, in particular psoriasis, by means of UV radiation with said device comprising a laser as well as a device for the most precise guidance of the laser beam.

Psoriasis is a skin disease widely spread in many countries from which about 3 million people suffer solely in Germany. For the treatment of this skin disease the UV light therapy in particular has proven its worth. While formerly to light sources were utilized that produced broad-band UVB rays it is common practice nowadays to make use of light sources only emitting longwave UVB radiation. In this manner undesirable side effects such as burns, skin aging and an increased risk of cancer can be diminished. Another possible treatment method is to apply the so-called PUVA therapy which instead of UVB light uses UVA light of longer wavelength in combination with a photosensitizing substance (psoralen).

A drawback with all these common light therapies is, however, that its application is not limited to the skin areas affected by the disease but also involves areas of healthy skin. In view of the risks for healthy skin associated with high doses of UVB radiation, in particular the elevated risk of cancer, it became necessary to develop a treatment method that enables selective skin areas to be irradiated most effectively and in a well aimed manner. Moreover, even if UV lamps having a narrow emission spectrum and a maximum of 311 nm were used as many of 25 to 40 treatment sessions were still required.

In the context of these considerations the use of a UVB excimer laser, as it is known in ophthalmology, has been reported and described in recent years (Bonis et al., Lancet. 1997; 350:1522). More exact studies in this field were carried out by Asawanonda et al., Arch. Dermatol. 2000; 136:619-24 as well as Feldman et al., J. Am. Acad. Dermatol. 2002; 46:900-6. In all these studies an XeCl excimer laser was used that emitted monochromatic UVB light of a wavelength of 308 nm which could be precisely directed at the skin areas to be treated.

In the studies hitherto performed the laser radiation dose that was applied to the individual plaques of skin areas affected by psoriasis was defined as a multiple of the so-called MED (minimal erythema dose). MED in this context is the minimal dose causing an erythema to develop, i.e. an inflammable abnormal redness of the skin, but without blister formation. This MED is determined initially on skin areas not affected by psoriasis before the actual treatment is started.

A typical treatment strategy here may thus involve that in the treatment of the psoriasis plaques initially 3 times the MED determined is applied when treatment commences which in the treatment sessions following initial treatment is increased or reduced depending on the individual treatment results. If it turns out, for example, that the plaques become thinner in the absence of a simultaneously occurring hyperpigmentation the dose has to be reduced by 1 MED to avoid the formation of blisters. On the other hand, if hyperpigmentation occurs the dose may be kept constant or increased further. In the event the affected skin area does not react the dose is to be increased by 1 MED, if sunburn or blisters are noted it shall be reduced by 1 MED.

Nevertheless, even at this state of the art there are still disadvantages in that the laser radiation dose cannot be optimally adapted to the individual patient. For instance, if there are patients with very thin plaques skin irritation even with blister formation may occur although a relatively low radiation dose has been applied whereas there are other patients who developed thicker-than-average plaques and must be exposed to a radiation dose 6 times MED to show a reaction. The reason for this is that the MED ascertained in skin areas external to those affected by the skin disease only relates very indirectly to the radiation dose required for the treatment of the psoriasis plaques. Aside from the risk that skin damage may occur the number of treatment sessions required will also increase unnecessarily for the patients which of course entails considerable stresses and higher costs as well.

Taking this state of the art into account there has been the objective to propose a method as well as a laser therapy device for the treatment of skin diseases, particularly psoriasis, that optimize the radiation dose to be applied to the individual skin areas affected by the disease thus allowing the treatment to be carried out in a gentle manner but at the same time expeditiously.

According to the invention this objective is reached by providing a method for the treatment of skin diseases, in particular psoriasis, with the aid of UV radiation generated by a laser and directed onto the skin areas affected by the disease with the thickness of the epidermis in such skin areas being determined and the laser radiation dose regulated depending on the epidermis thickness so detected, as well as by a laser therapy device for the treatment of skin diseases, particularly psoriasis, using UV radiation produced by means of a laser and a device for the most precise guidance of the laser beam wherein the laser therapy device is equipped with a control mechanism that automatically regulates the laser radiation dose applied, for curing purposes, to a skin area affected by the skin disease depending on the epidermis thickness found in such skin areas.

The invention is based on the knowledge that the effect of UV radiation on the affected skin areas where psoriasis has developed into so-called plaques is primarily governed by the thickness of the epidermis within such plaques and not by the skin type or the MED established for healthy skin regions. Comprehensive studies have therefore been carried out to establish the interrelation between the thickness of the epidermis and the radiation dose that is just sufficient to cause skin redness within the skin area affected by psoriasis. This latter radiation dose was called MED-I (minimal erythema dose of the involved skin) and is thus the radiation dose to be applied when treatment of a plaque starts. This MED-I increases basically linearly as a function of the thickness of the epidermis. Thus, an epidermis thickness of 200 μm is linked to an MED-I dose of approx. 600 mJ/cm² whereas a radiation dose in the range of 1100 mJ/cm² is considered reasonable for an epidermis thickness of 400 μm.

It is to be observed in this context that the epidermis thickness may vary with the respective plaque to be treated so that laser radiation doses should preferably be determined in each individual case to achieve optimum treatment results. Naturally, such a selective treatment of individual plaques is not possible with the methods known from prior art which require that a uniform MED value is to be determined on the basis of healthy skin areas.

The laser therapy device according to the invention provides for a laser generating a beam capable of being most precisely directed to the skin areas to be treated so that it is warranted that skin regions not affected by the disease will not be impaired by the treatment. This laser therapy device automatically regulates the laser radiation dose with the help of a control depending on the thickness of the epidermis of the skin area to be actually treated, for which purpose common software known to those skilled in the art can be employed. Determination of the epidermis and selecting the radiation dose based on this determination is preferably effected for each individual plaque; nevertheless it is also possible, especially where plaques in great number are involved, to just determine the epidermis thickness with respect to individual plaques and then keep the radiation dose constant when treating several plaques found within a skin area.

A device for the determination of the thickness of the epidermis preferably forms an integral part of the laser therapy device and is connected directly with the control system so that the radiation dose can be adapted automatically and the person conducting the treatment is not required to make further settings. This enables the therapy to be performed easier and quicker and, moreover, highly qualified personnel for the control of the system will not be needed which furthermore results in cost savings. In particular, the device and laser unit may directly be integrated in a single housing and thus form integral parts of a common system.

For the purpose of determining the thickness of the epidermis an ultrasonic device is especially suited by means of which an accurate, quantitative, non-invasive examination of the skin can be performed. Typically, ultrasonic measurements will be effected at a frequency of approx. 20 MHz. Such an ultrasonic device is, for example, available from taberna pro medicum GmbH, of Lüneburg, Germany.

For the method according to the invention an excimer laser is preferably employed which emits an especially intensive light in the ultraviolet spectral range. Particularly suited is an XeCl laser operating at a wave length of 308 nm.

Such excimer lasers may, for example, be procured from TUI-Laser AG, Munich, Germany. A device is on the market under the tradename of STELLA® that operates at a pulse repetition rate of 200 Hz and a pulse duration of 60 ns. The energy per pulse amounts to 4 mJ with the energy capable of being concentrated on a certain treatment area. Given a treatment area of 2 cm² this results in an energy density of 2 mJ/cm². The radiation dose can be varied between 100 and 6000 mJ/cm² in increments of 50 mJ/cm².

The well-aimed transmission of the radiation and the most precise direction and guidance on to certain areas of the skin is preferably brought about with the aid of a flexible light conductor which is equipped with an end piece to be placed on the skin area to be treated. Since the element is directly placed on the skin areas subjected to the treatment it is almost ruled out that non-affected skin regions are exposed to radiation. In the area of the end piece an aperture of square shape may be employed so that the treatment, to a large extent, can take place without overlaps.

In particular the end piece of the light conductor and the ultrasonic probe, also intended to be placed on the skin, connected to the ultrasonic device may also be combined to form a single unit. This is especially advantageous in that as soon as the thickness of the epidermis in the area of a given plaque has been determined the laser therapy device according to the invention will automatically decide on the laser radiation dose to be used and applied immediately to the plaque. In this way, a tailored treatment of each individual plaque will be greatly facilitated.

As an alternative to the use of a light conductor directing the laser beam precisely at certain skin areas a mirror arm may be employed by means of which the light is guided with the help of a system usually comprising several mirrors.

As per another configuration of the method and/or laser therapy device according to the invention the MED-I value that causes a visible redness without blister formation in the plaque region is additionally and directly measured at least in some skin areas with said value then being used in conjunction with the determined epidermis thickness to regulate the laser radiation dose applied to the skin area in question. The MED-I value may be determined by the laser therapy device or separately and, if appropriate, introduced to the laser therapy device. In this manner the interrelation between MED-I value and epidermis thickness is determined specifically for the individual patient so that an exceptional reaction of a patient's skin that may possibly differ from that of the average patient can be duly taken into account. Particularly in cases where the MED-I value differs significantly from the average MED-I value established for the epidermis thickness detected the laser radiation dose used in the treatment may appropriately be increased or reduced as deemed applicable.

Another approach that can be taken in this context is to determine just for a few skin areas the MED-I value in conjunction with the respective epidermis thickness; for the majority of plaques, however, only the epidermis thickness is determined. This may then be correlated with the epidermis thickness of the skin area for which a MED-I value has previously been established so that it is possible, particularly through the control of the laser therapy device, to simulate a MED-I value for all skin areas to be treated which is then used as radiation dose in the respective treatment. Such a simulation can be easily effected particularly because the interrelation between the MED-I value and the epidermis thickness is primarily a linear one. The laser therapy device therefore comprises an internal calibration feature that can be used to vary the radiation dose as a function of the epidermis thickness.

Moreover, provisions can be taken to adapt the laser radiation dose in treatment sessions following initial treatment to take into account the treatment success hitherto achieved. In this way the radiation dose may be increased step by step for individual plagues that do not show a visible reaction or where hyperpigmentation occurs. Should this not be the case, the radiation dose is to be kept constant.

Another preferred possibility to adapt the radiation dose to the success of the treatment is to newly determine the thickness of the epidermis of a skin area affected by the skin disease after each individual treatment with UV laser radiation and depending on the relevant result newly fix the laser radiation dose for the next treatment session. As the treatment progresses it is to be expected that the plaques become thinner so that the laser radiation dose can be appropriately reduced to make sure an as gentle as possible skin treatment is achieved. Proceeding in this manner will ensure that, on the one hand, when at the beginning of the treatment the plaques are very thick a sufficiently great radiation dose is applied in order to achieve a reasonable treatment success and, on the other, will prevent the skin from being subjected to undue stresses by unnecessarily high doses of radiation towards the end of the treatment sessions when plaques have become significantly thinner.

Even if the method according to the invention as well as the inventive laser therapy device has been described herein with emphasis on its application for the treatment of psoriasis it is of course possible and obvious for those skilled in the art that it may likewise be employed for the treatment of other skin diseases. A laser therapy device for the treatment of such other skin diseases shall for that reason not at all be excluded from the protective scope. Such other disease shall in particular include vitiligo, neurodermitis, acne, repigmentation of scars, repigmentation of hypopigmented skin areas after skin resurfacing, mycosis funguides, exantematic lichen ruber, granuloma anulare, lichen planus or alopecia areata.

Further elucidation of the invention is provided through the enclosed figures, where

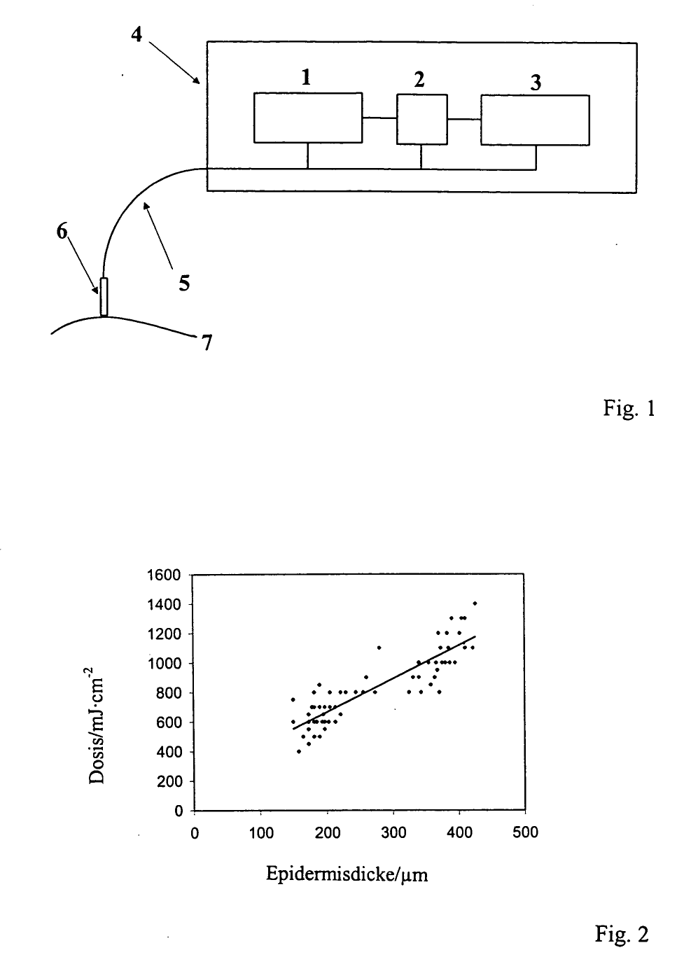

FIG. 1 shows a schematic configuration of the laser therapy device according to the invention;

FIG. 2 is a graphical representation explaining the interrelation between MED-I and epidermis thickness.

In FIG. 1 the configuration of the laser therapy device according to the invention is shown schematically. The control unit 2 of the laser therapy device is connected to both a laser 1 and to an ultrasonic device 3 with the separate representation of these units serving the sole purpose of providing clarification. In this case these units are accommodated in housing 4. The laser 1 is provided with a light conductor 5 fitted at one end with end piece 6 with aperture which can be placed exactly onto the skin areas 7 to be treated. Furthermore, an ultrasonic probe is integrated into end piece 6 with said probe being connected with the ultrasonic device 3 by means of a cable arranged parallel to light conductor 5. As a result of this, the combined end piece 6 needs to be placed only once on the skin area 7 to be treated to determine the thickness of the epidermis, directly calculate the radiation dose and apply this dose to the skin. In this manner, the use and operation of the device is further facilitated.

FIG. 2 illustrates the MED-I (minimal erythema dose in involved skin) characteristic shown versus the thickness of the epidermis. In this figure MED-I represents the dose that causes an erythema, i.e. a skin redness, to form within a plaque without blisters developing. The thickness of the epidermis was determined with the aid of a 20-MHz ultrasonic device. As can be seen from the graphical representation MED-I increases as a function of the epidermis thickness with such correlation being, on average, a linear one. It is also evident from said figure that there is a significant variance from patient to patient so that it appears quite expedient to have available an individual correction means to vary the radiation dose applied on the basis of a determined MED-I value by correlating said value with the epidermis thickness in the way described above.

### EXAMPLE

In a test carried out as per the inventive treatment method 40 psoriasis patients were subjected to the above mentioned treatment. The therapy started with a radiation dose at MED-I level being applied initially wherein the MED-I value was correlated with the epidermis thickness determined by an ultrasonic method. For the determination of the epidermis thickness a 20-MHz ultrasonic system was used. Setting the laser radiation dose was performed manually in this case making use of external devices.

From the initial 40 patients who started the therapy 37 were able to finalize it whereas 3 patients had to give up for various reasons. The initial radiation dose amounted to 797 mJ/cm²±231 mJ/cm² which corresponds to 2.6 to 7 times the MED value established by conventional methods. In 14.8% of the cases the formation of blisters could be observed during treatment. A decline of the plaques by 90% or more was detected at the end of the therapy in 83.7% of the cases treated. This result was achieved after 7.1 treatments on average and a cumulated total radiation dose of 6,254 mJ/cm². In a comparison group examined where the initial radiation dose was not tailored to the needs of the individual patient a comparable treatment result could only be obtained after 13 single treatments on average and a cumulated total radiation dose of 11,250 mJ/cm². This translates into a reduction of the total radiation dose by more than 40%. At the same time, side effects in the form of blister formation occurred in 40% of the cases, i.e. this occurred twice as often as experienced with a treatment carried out with the laser therapy device according to the invention.

1. Method for the treatment of skin diseases with the aid of UV radiation generated by a laser and directed onto the skin areas affected by the disease wherein the thickness of the epidermis in such skin areas is determined and the laser radiation dose regulated depending on the epidermis thickness so detected.

2. Method according to claim 1 wherein the thickness of the epidermis is determined individually for each affected skin area and depending on this thickness the laser radiation dose is individually regulated for each affected skin area.

3. Method according to claim 1 wherein the laser radiation dose applied during treatment is increased in subsequent treatment sessions when a hyperpigmentation occurs within the treated skin area or in the event a visible reaction cannot be noted, and should this not be the case is maintained.

4. Method according to claim 1 wherein the thickness of the epidermis of a skin area affected by the skin disease is newly determined after a treatment by means of UV radiation and, based on this, the laser radiation dose being applied during the next treatment is newly adapted.

5. Method according to claim 1 wherein an ultrasonic device is employed for the determination of the epidermis thickness.

6. Method according to claim 1 wherein an excimer laser is employed as laser device.

7. Method according to claim 6 wherein a XeCl laser is employed as excimer laser.

8. Method according to claim 1 wherein a flexible light conductor provided with an end piece for the placement onto the skin areas to be treated is used for the direction of the UV radiation to skin areas affected by the skin disease.

9. Method according to claim 8 wherein the end piece of the light conductor is combined into an integral unit formed with an ultrasonic probe connected with an ultrasonic device for the determination of the thickness of the epidermis.

10. Method according to claim 1 wherein a mirror arm is employed for the direction of the UV radiation onto the skin areas affected by the skin disease.

11. Method according to claim 1 wherein for the implementation of the method a laser therapy device with a control system is used with said control automatically regulating the laser radiation dose to be applied to skin areas affected by the skin disease as a function of the thickness of the epidermis of these skin areas.

12. Method according to claim 1 wherein the method is used for the treatment of psoriasis, vitiligo, neurodermitis, acne, mycosis funguides, exantematic lichen ruber, granuloma anulare, lichen planus, alopecia areata, or for the repigmentation of scars or hypopigmented skin areas after skin resurfacing.

13. Method according to claim 1 wherein at least in some of the skin areas affected by the skin disease the laser radiation dose is determined that causes a visible redness without blister formation to occur and based on this given laser radiation dose and the thickness of the epidermis detected in this skin area the laser radiation dose for the treatment of this skin area is regulated.

14. Method according to claim 13 wherein the thickness of the epidermis of various skin areas affected by the skin disease is correlated with the thickness of the epidermis of one skin area for which the laser radiation dose causing a visible redness without blister formation has been determined and based on said thickness the laser radiation dose to be applied for treatment is individually established for each individual skin area to be treated.

15. Method according to claim 13 wherein the laser radiation dose applied during treatment is increased in subsequent treatment sessions when a hyperpigmentation occurs within the treated skin area or in the event a visible reaction cannot be noted, and should this not be the case is maintained.

16. Method according to claim 13 wherein the thickness of the epidermis of a skin area affected by the skin disease is newly determined after a treatment by means of UV radiation and, based on this, the laser radiation dose being applied during the next treatment is newly adapted.

17. Method according to claim 13 wherein an ultrasonic device is employed for the determination of the epidermis thickness.

18. Method according to claim 13 wherein an excimer laser is employed as laser device.

19. Method according to claim 18 wherein a XeCl laser is employed as excimer laser.

20. Method according to claim 13 wherein a flexible light conductor provided with an end piece for the placement onto the skin areas to be treated is used for the direction of the UV radiation to skin areas affected by the skin disease.

21. Method according to claim 20 wherein the end piece of the light conductor is combined into an integral unit formed with an ultrasonic probe connected with an ultrasonic device for the determination of the thickness of the epidermis.

22. Method according to claim 13 wherein a mirror arm is employed for the direction of the UV radiation onto the skin areas affected by the skin disease.

23. Method according to claim 13 wherein for the implementation of the method a laser therapy device with a control system is used with said control automatically regulating the laser radiation dose to be applied to skin areas affected by the skin disease as a function of the thickness of the epidermis of these skin areas.

24. Method according to claim 13 wherein the method is used for the treatment of psoriasis, vitiligo, neurodermitis, acne, mycosis funguides, exantematic lichen ruber, granuloma anulare, lichen planus, alopecia areata, or for the repigmentation of scars or hypopigmented skin areas after skin resurfacing.

25. Laser therapy device for the treatment of skin diseases with the aid of UV radiation comprising a laser and a device for the most precise guidance of the laser beam, wherein the laser therapy device is equipped with a control mechanism that automatically regulates the laser radiation dose applied, for curing purposes, to a skin area affected by the skin disease depending on the epidermis thickness found in such skin areas.

26. Laser therapy device according to claim 25 wherein the laser therapy device comprises a device for the determination of the thickness of the epidermis in skin areas affected by the skin disease.

27. Laser therapy device according to claim 26 wherein the device for the determination of the thickness of the epidermis and the laser are integrated into a single housing.

28. Laser therapy device according to claim 26 wherein the device for the determination of the epidermis thickness is an ultrasonic device.

29. Laser therapy device according to claim 25 wherein an excimer laser is employed as laser device.

30. Laser therapy device according to claim 29 wherein a XeCl laser is employed as excimer laser.

31. Laser therapy device according to claim 25 wherein the device for the most precise guidance of the laser beam is a flexible light conductor with end piece to be placed onto the skin area to be treated.

32. Laser therapy device according to claim 31 wherein an ultrasonic probe connected with an ultrasonic device and the end piece of the light conductor are combined to form an integral unit.

33. Laser therapy device according to claim 25 wherein the device for the most precise guidance of the leaser beam is a mirror arm.

34. Laser therapy device according to claim 25 wherein the laser radiation dose applied during treatment is increased in subsequent treatment sessions when a hyperpigmentation occurs within the treated skin area or in the event a visible reaction cannot be noted, and should this not be the case is maintained.

35. Laser therapy device according to claim 25 wherein the laser therapy device newly determines the thickness of the epidermis of a skin area affected by the skin disease after a treatment by means of UV radiation and, based on this, newly adapts the laser radiation dose being applied during the next treatment.

36. Laser therapy device according to claim 25 wherein the laser therapy device determines in individual skin areas affected by the skin disease the laser radiation dose that causes a visible redness without blister formation to occur, or that this laser radiation dose is determined separately, and that the laser therapy device based on the laser radiation dose so determined and the thickness of the epidermis detected in this skin area regulates the laser radiation dose for the treatment of this skin area.

37. Laser therapy device according to claim 36 wherein the laser therapy device correlates the thickness of the epidermis of various skin areas affected by the skin disease with the thickness of the epidermis of one skin area for which the laser radiation dose causing a visible redness without blister formation has been determined and based on said thickness establishes individually for each individual skin area to be treated the laser radiation dose to be applied for treatment.

38. Laser therapy device according to claim 36 wherein the laser therapy device comprises a device for the determination of the thickness of the epidermis in skin areas affected by the skin disease.

39. Laser therapy device according to claim 38 wherein the device for the determination of the thickness of the epidermis and the laser are integrated into a single housing.

40. Laser therapy device according to claim 38 wherein the device for the determination of the epidermis thickness is an ultrasonic device.

41. Laser therapy device according to claim 36 wherein an excimer laser is employed as laser device.

42. Laser therapy device according to claim 41 wherein a XeCl laser is employed as excimer laser.

43. Laser therapy device according to claim 36 wherein the device for the most precise guidance of the laser beam is a flexible light conductor with end piece to be placed onto the skin area to be treated.

44. Laser therapy device according to claim 43 wherein an ultrasonic probe connected with an ultrasonic device and the end piece of the light conductor are combined to form an integral unit.

45. Laser therapy device according to claim 36 wherein the device for the most precise guidance of the laser beam is a mirror arm.

46. Laser therapy device according to claim 36 wherein the laser radiation dose applied during treatment is increased in subsequent treatment sessions when a hyperpigmentation occurs within the treated skin area or in the event a visible reaction cannot be noted, and should this not be the case is maintained.

47. Laser therapy device according to claim 36 wherein the laser therapy device newly determines the thickness of the epidermis of a skin area affected by the skin disease after a treatment by means of UV radiation and, based on this, newly adapts the laser radiation dose being applied during the next treatment.
